(19) United States
(12) Patent Application Publication
(10) Pub. No.: US 2024/0272173 A1
(43) Pub. Date: Aug. 15, 2024

(54) Bioanalysis Workflow with Direct Ejection from Phase-Separated Samples

(71) Applicant: DH Technologies Development Pte. Ltd., Singapore (SG)

(72) Inventors: Thomas COVEY, Concord (CA); Yuzhu GUO, Kleinburg (CA); Chang LIU, Richmond Hill (CA)

(21) Appl. No.: 18/568,398

(22) PCT Filed: Jun. 8, 2022
(86) PCT No.: PCT/IB2022/055351

§ 371 Date: Dec. 8, 2023

Related U.S. Application Data
(60) Provisional application No. 63/208,696, filed on Jun. 9, 2021.

(51) Int. Cl. G01N 33/68; G01N 30/06; G01N 30/72

(52) U.S. Cl. CPC G01N 33/6851; G01N 30/06; G01N 30/7233

(57) ABSTRACT

In one aspect, a method of introducing a sample into an open port interface (OPI) of a mass spectrometer is disclosed, which includes mixing the sample with a solvent in which a matrix of an aqueous phase of the sample is immiscible and in which at least a target analyte, when present in the sample, is miscible so as to extract at least a portion of the target analyte into said at least one solvent, thereby generating a multi-phase liquid having said aqueous phase and one or more organic phases, wherein at least one of those organic phases contains at least a portion of the target analyte. In some embodiments, the method further calls for ejecting a plurality of droplets from at least one of the phases of the multi-phase liquid for introduction into the OPI of the mass spectrometer.

### BACKGROUND

The present disclosure is directed generally to systems and methods for mass spectrometry, and more particularly to such systems and methods for introducing samples into mass spectrometers.

Mass spectrometry (MS) is an analytical technique for determining the structure of test chemical substances with both qualitative and quantitative applications. MS can be useful for identifying unknown compounds, determining the composition of atomic elements in a molecule, determining the structure of a compound by observing its fragmentation, and quantifying the amount of a particular chemical compound in a mixed sample. Mass spectrometers detect chemical entities as ions such that a conversion of the analytes to charged ions must occur during the sampling process.

LC-MS (liquid chromatography-mass spectrometry) is an analytical technique in which an LC column is employed upstream of a mass spectrometer to separate different analytes in a sample according to their retention times in the LC column for time-separated introduction into the mass spectrometer for chemical analysis.

Thus, there is a need for enhanced systems for introducing samples into mass spectrometers.

### SUMMARY

In one aspect, a method of introducing a sample into a mass spectrometer is disclosed, which includes mixing the sample with a solvent in which a matrix of an aqueous phase of the sample is immiscible and in which at least a target analyte, when present in the sample, is miscible so as to extract at least a portion of the target analyte into said at least one solvent, thereby generating a multi-phase liquid (herein also referred to as a multi-phase system) having said aqueous phase and one or more organic phases, wherein at least one of said organic phases contains at least a portion of the target analyte. The method further calls for introducing one or more samples from at least one of the phases of the multi-phase liquid, e.g., the phase into which the target analyte is extracted, into the mass spectrometer, e.g., via the ejection of one or more liquid droplets thereof into an OPI of the mass spectrometer.

In some embodiments, an acoustic actuator can be employed to eject one or more droplets from an organic phase that contains at least a portion of the target analyte into the mass spectrometer. Alternatively, the plurality of droplets can be ejected from any of the other organic phases, if any, to introduce, e.g., one or more analytes contained in those organic phases into the mass spectrometer. Further, in some embodiments, the plurality of the droplets can be ejected from the aqueous phase into the mass spectrometer, e.g., to introduce a target analyte contained in the aqueous phase into the mass spectrometer.

A variety of mechanisms can be employed for introducing one or more samples of at least one of the phases of the multi-phase liquid into the OPI of the mass spectrometer. By way of example, in some embodiments, one or more acoustic pulses can be applied to at least one of the phases (e.g., the aqueous or any of the organic phases) to cause one or more samples of that phase (e.g., in the form of a plurality of droplets) to be introduced into the mass spectrometer. In some embodiments, several phases of the multi-phase liquid may be stacked in a sample holder along a vertical direction in accordance with their respective densities. In some such embodiments, an ejection mechanism (e.g., an acoustic actuator) can be operably coupled to the sample holder in order to selectively eject one or more samples of any of those phases into the mass spectrometer.

By way of example, the ejection mechanism may be coupled to the top of the sample holder to eject one or more droplets of the top phase into the mass spectrometer. Alternatively, the ejection mechanism may be coupled to the bottom of the sample holder in order to eject a liquid phase at the bottom of the sample holder into the mass spectrometer.

In some embodiments, an aqueous sample may be mixed with a single organic solvent for generating a two-phase liquid sample. In other embodiments, an aqueous sample may be mixed with a plurality of organic solvents (e.g., two or more organic solvents) for generating a multi-phase liquid sample. By way of example, the plurality of organic solvents can include two different organic solvents.

By way of example, in some embodiments, a plurality of organic solvents (e.g., organic solvents) may be added to an aqueous solution containing a plurality of target analytes of interest such that at least one of those target analytes is miscible in one of the organic solvents and at least another one of the target analytes is miscible in another one of the organic solvents so as to extract (at least partially) those target analytes into different ones of the organic solvents. In this manner, a plurality of target analytes of interest can be extracted into different organic solvents and each target analyte can be selectively introduced into a mass spectrometer via sampling the respective phase in which that target analyte is contained (e.g., via a droplet ejection mechanism).

In some embodiments, the aqueous phase can be more dense than one or more organic phases while in other embodiments the aqueous phase may be less dense than one or more of the organic phases of the multi-phase liquid.

By way of example, in some embodiments in which an organic phase can include a mixture of the two organic solvents, the solvents can be hexane and methyl acetate. In some such embodiments, the aqueous phase can include a mixture of acetonitrile and water. By way of example, in some such embodiments, the relative concentration ratios of the hexane, the methyl acetate, the acetonitrile and the water can be 4:4:3:4.

As noted above, in some embodiments, the aqueous phase can be more dense than the organic phase. For example, in some such embodiments, the organic phase can include any of chloroform, dichloromethane, ethyl ether and combinations thereof.

As noted above, in some embodiments, one or more acoustic pulses can be applied to any of the aqueous or the organic phases for the introduction of one or more samples thereof into a mass spectrometer for measuring at least one mass signal thereof. However, the practice of the present teachings is not limited to the use of acoustic pulses. For example, in some embodiments, one or more controlled pressure pulses can be applied to a bottom phase of a multi-phase liquid so as to eject one or more droplets of that phase through a small opening provided at the bottom a sample holder into a mass spectrometer.

Further understanding of various aspects of the present teachings can be obtained by reference to the following detailed description in conjunction with the associated drawings, which are described briefly below.

### BRIEF DESCRIPTION OF THE DRAWINGS

FIG. 1 shows a flow chart depicting various steps according to an embodiment for introducing one or more analytes into an open port interface (OPI) of a mass spectrometer,

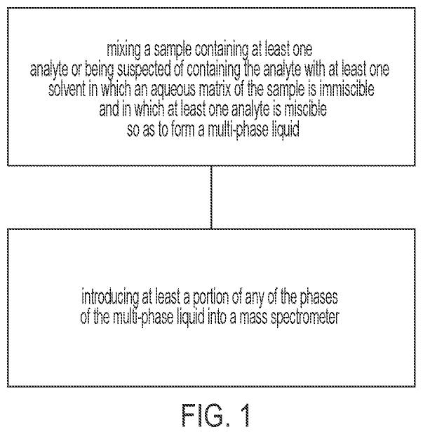

FIG. 2 schematically depicts a two-phase system comprising an upper organic phase and a lower aqueous phase,

FIG. 3 schematically depicts a three-phase system comprising an aqueous phase and two phase-separated organic phases,

FIG. 4 is an example of a mass spectrometric system according to an embodiment, which is equipped with an acoustic actuator for ejecting samples of at least one phase of a multi-phase system prepared according to embodiments of the present teachings into a mass spectrometer of the system,

FIGS. 5A, 5B, and 5C depict measurements of a mass signal associated with dextromethorphan acquired from a urine sample without using organic phase extraction of dextromethorphan from the urine sample,

FIGS. 6A, 6B, and 6C depict measurements of the same mass signal of dextromethorphan acquired from an organic phase of a two-phase system, where the dextromethorphan was extracted from a urine sample into the organic phase, and

FIG. 7 schematically illustrates an example of implementation of a controller in accordance with embodiments of the present teachings.

### DETAILED DESCRIPTION

It will be appreciated that for clarity, the following discussion will explicate various aspects of embodiments of the present disclosure, while omitting certain specific details wherever convenient or appropriate to do so. For example, discussion of like or analogous features in alternative embodiments may be somewhat abbreviated. Well-known ideas or concepts may also for brevity not be discussed an any great detail. One of ordinary skill will recognize that some embodiments of the present disclosure may not require certain of the specifically described details in every implementation, which are set forth herein only to provide a thorough understanding of the embodiments. Similarly it will be apparent that the described embodiments may be susceptible to alteration or variation according to common general knowledge without departing from the scope of the disclosure. The following detailed description of embodiments is not to be regarded as limiting the scope of the applicant's teachings in any manner.

As used herein, the terms “about” and “substantially equal” refer to variations in a numerical quantity that can occur, for example, through measuring or handling procedures in the real world; through inadvertent error in these procedures; through differences in the manufacture, source, or purity of compositions or reagents; and the like. Typically, the terms “about” and “substantially” as used herein means 10% greater or lesser than the value or range of values stated or the complete condition or state. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

As used herein the term “and/or” includes any and all combinations of one or more of the associated listed items and may be abbreviated as “/”.

As used herein and conventionally understood, two substances are “miscible” when they can be mixed in all proportions to form a homogeneous mixture. For example, water and alcohol are miscible because they can be mixed in all proportions to form a homogenous mixture. Further, two substances are immiscible when their mixing does not result in a homogeneous mixture.

The term “aqueous phase” as used herein refers to a medium that contains water. An aqueous solution refers to a solution in which the solvent is water. The term “organic phase” refers to a medium that does not form a homogeneous mixture when mixed with an aqueous phase.

LC-MS is becoming an important analytical tool in clinical and drug development for the measurement of target analytes in complex biological samples (e.g., urine, blood, etc.) due to its high specificity relative to immunoassays. LC-MS workflow requires sample preparation for cleaning the sample, for example, in order to minimize ionization suppression that could occur from hydrophilic residual matrix components, such as inorganic salts, and to maintain the LC column and the mass spectrometer as clean as possible.

Some common methods for sample preparation include protein precipitation and/or solid phase extraction (SPE) methods. These techniques require multiple additional sample handling steps, thereby reducing the overall analytical throughput of the system. Further, the inventors have realized that in some cases, the matrix of a conventionally prepared sample may lead to ion suppression of a target analyte of interest, thereby adversely affecting an acquired mass signal of that analyte.

The present disclosure relates to methods and systems for introducing one or more analytes into an open port interface (OPI) of a mass spectrometer.

In some mass spectrometers, acoustic pulses can be applied to a sample to generate sample droplets for introduction into an ion source of a mass spectrometer. For example, in AEMS (Acoustic Ejection Mass Spectrometry) systems, acoustic pulses are applied to a sample to eject droplets into an ion source of a mass spectrometer. The AEMS systems provide a much faster sample readout relative to LC-MS systems. In addition, AEMS systems allow low volume sample ejection (e.g., nanoliters of samples) and a high degree of dilution (e.g., up to several thousand folds) into an OPI (Open Port Interface) of a mass spectrometer, thereby reducing ionization suppression for some matrices. AEMS systems further simplify workflow development and sample preparation and improve the overall analytical throughput.

However, inventors have discovered that a certain level of ionization suppression may still be observed for some complex matrices, especially when multi-drop scale sample loading is used, which can reduce assay sensitivity.

The present disclosure generally relates to methods and systems for analyzing samples, and in particular, for analyzing samples having complex matrices based on direct sampling from a multi-phase system (e.g., a two-phase system). In embodiments, the present disclosure provides methods and systems that allow high-throughput sample introduction into a mass spectrometer by generating a multi-phase system (e.g., a two-phase system) in which a target analyte has been extracted at least partially from one phase into another phase, e.g., from an aqueous phase into an organic phase.

In some embodiments, the samples are biological samples, though the present teachings are not limited to such samples. By way of example, and as discussed in more detail below, a solvent (e.g., Pentanol, Octanol, etc.) can be added to a biological sample containing, or being suspected of containing, a target analyte that is miscible in the solvent so as to extract the target analyte (or at least a portion thereof) into the solvent while leaving behind an aqueous sample matrix (e.g., salts or other analytes) in the aqueous phase. In some embodiments, one or more samples of the solvent into which the target analyte is extracted can be introduced into a mass spectrometer to acquire one or more mass signals of the target analyte. In many embodiments, such liquid-liquid-extraction (LLE) system can be employed for direct introduction of the target analyte into a mass spectrometer without any additional sample preparation steps. As discussed below, in many embodiments, such an approach can advantageously result in a significant reduction in ionization suppression, thus improving the assay sensitivity.

By way of illustration, an embodiment of a method according to the present teachings for introducing one or more analytes into an open port interface (OPI) of a mass spectrometer includes mixing a sample containing one or more target analytes or being suspected of containing one or more target analytes with a solvent in which an aqueous matrix of the sample is immiscible and in which at least one of those target analytes is miscible so as to extract at least one of the analytes (or at least a portion of said at least one of the analytes) into the solvent, thereby generating a two-phase liquid having a first phase comprising the aqueous matrix and a second phase comprising a mixture of the solvent and at least a portion of said at least one of said analytes (step 1).

One or more droplets can be ejected from the second phase for introduction into the OPI of the mass spectrometer (step 2). In some embodiments, such ejection of one or more sample droplets can be achieved via application of one or more acoustic pulses to an exposed surface of the sample, although the present teachings are not limited to the use of acoustic pulses for introduction of one or more samples of any of the aqueous and organic phases into a mass spectrometer.

By way of example, in some embodiments, a phase-separated system can be generated by adding an organic solvent to an aqueous sample containing or suspected of containing of one or more target analytes of interest such that at least one of those analytes, when present in the sample, is extracted from the aqueous sample into the organic solvent.

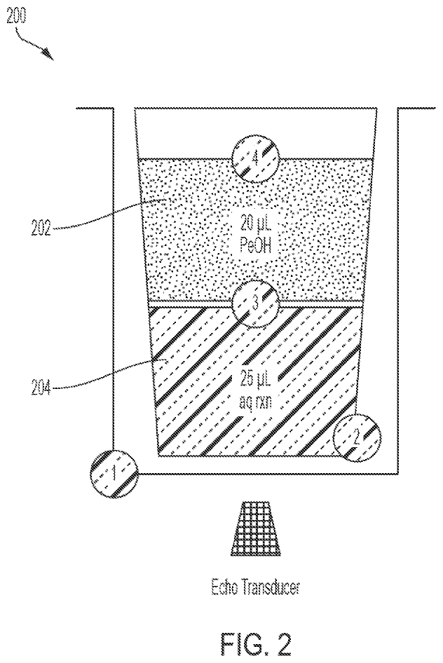

By way of illustration, FIG. 2 schematically depicts a two-phase system 200 that is prepared by adding a quantity of Pentanol 202 to an aqueous layer 204 containing a mixture of a target analyte and water, where the target analyte is miscible in Pentanol. The addition of the Pentanol layer to the aqueous layer results in the extraction of at least a portion of the target analyte contained in the aqueous layer into the Pentanol layer. In this embodiment, an acoustic actuator 206 can be coupled to the Pentanol layer so as to eject one or more samples of the Pentanol layer containing the target analyte into a mass spectrometer (not shown in this figure).

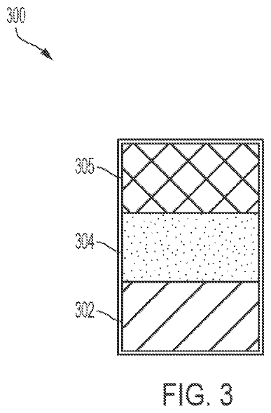

In other embodiments, the multi-phase liquid includes more than two phases. By way of illustration, FIG. 3 schematically depicts a three-phase liquid 300 that includes an aqueous phase 302 and two organic phases 304 and 305. By way of example, in some embodiments, the organic phase 304 can be chloroform and the organic phase 305 can be Pentanol (PeOH).

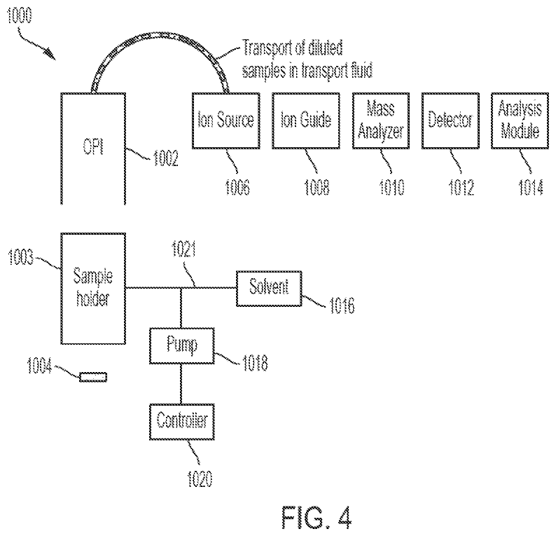

As shown schematically in FIG. 4, in some embodiments, a mass spectrometer 1000 having an open port interface (OPI) 1002 can be employed for receiving one or more samples of at least one phase of a multi-phase system prepared in accordance with the present teachings for providing a mass analysis of a target analyte contained in that phase.

As shown schematically in FIG. 4, in this embodiment, a sample holder 1003 (herein also referred to as a “first reservoir”) can receive a sample of interest (such as a biological sample). A solvent reservoir 1016 (herein also referred to as a “second reservoir”) can store at least one solvent, e.g., an organic solvent, for extracting a target analyte of interest from the sample of interest. A fluid path 1021 can fluidly couple the solvent reservoir to the sample holder 1003. A pump 1018 operating under control of a controller 1020 is fluidly coupled to the fluid path 1021 to facilitate the transfer of the solvent(s) from the solvent reservoir to the sample holder. In some embodiments, the controller 1020 can be programmed to control the pump for transferring a predefined volume of the solvent from the second reservoir to the first reservoir.

The mixing of the organic solvent with the sample can result in extraction of at least a portion of the target analyte from an aqueous matrix of the sample into the solvent, as discussed above, thus forming a multi-phase system.

An acoustic droplet dispenser 1004 (herein also referred to as an acoustic actuator) is operably coupled to the sample holder 1003 for introducing one or more samples of at least one phase of the multi-phase system (in this embodiment, the organic phase containing the extracted target analyte) into the open port interface (OPI) 1002 of the mass spectrometer.

In this embodiment, the OPI interface 1002 carries the received droplets containing the target analyte to an atmospheric pressure ionization source (API) source 1006, which ionizes at least a portion of the target analyte so as to generate a plurality of ions. The ions are received by an ion guide 1008 that focuses the ions to generate an ion beam, which is received in turn by at least one downstream mass analyzer 1010 (e.g., one or more quadrupole and/or time-of-flight mass analyzers).

At least a portion of the ions passing through the mass analyzer is received by a downstream ion detector 1012, which generates ion detection signals in response to the detection of the ions. An analysis module 1014 in communication with the ion detector 1012 can receive the ion detection signals generated by the ion detector and can generate a mass spectrum of the target ions and/or fragments thereof. By way of example, in some embodiments, the mass spectrometer 1000 can operate in multiple reaction monitoring (MRM) mode in which a plurality of precursor target ions can be selected via a mass analyzer and can be fragmented in a downstream collision cell to generate a plurality of product ions, where the product ions are detected via the ion detector, which generates ion detection signals that can be analyzed by the analysis module in a manner known in the art.

Any of the phases of a multi-phase system can be ejected into the mass spectrometer. By way of example, in some embodiments, the acoustic actuator can eject one or more droplets via application of one or more acoustic pulses to an exposed surface of a top layer of the multi-phase system into the mass spectrometer. Alternatively, a sample ejection system configured for ejecting one or more samples of a phase layer positioned at the bottom of the sample holder can be employed for introducing one or more samples of that layer into the mass spectrometer.

By way of example, a sample ejection system marketed by CellLink Life Sciences of Phoenix, U.S.A. under the trade designation i-Dot (Immediate Drop-on Demand Technology) can be employed for ejecting one or more samples of a bottom phase layer of a multi-phase system into the mass spectrometer. The i-Dot system employs a plurality of positive controlled pressure pulses to generate droplets with volumes in a range of about 8 to about 50 nanoliters from a small opening at the bottom of a sample holder, e.g., a well in which a multi-phase system according to the present is stored.

In some embodiments in which the sampling of a middle phase of a multi-phase system is desired, one or more of the phases can be depleted to make the target middle phase as the top or the bottom phase. By way of example, this can be followed by ejecting one or more droplets of the target phase, e.g., in a manner discussed above in connection with sampling a top or a bottom phase of a multi-phase system.

Alternatively, in some embodiments in which the sampling of a middle phase of a three-phase system is desired, acoustic energy can be focused at the interface between the top and the middle layer to generate one or more droplets from the middle layer. Such droplet(s) can propagate through the top layer and be captured by an OPI of a mass spectrometer. In some such embodiments, the droplets of the middle sample arriving at the OPI may be coated with a thin layer of the medium of the top layer.

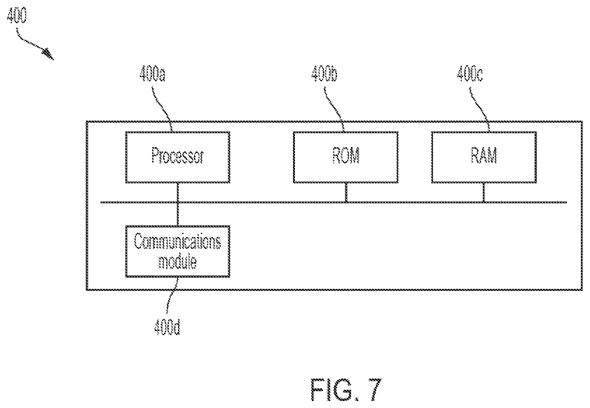

The controller 1020 for operating the pump 1018 can be implemented in hardware, software and/or firmware in a manner known in the art as informed by the present teachings. By way of example, FIG. 7 schematically depicts an example of an implementation of such a controller 400, which includes a processor 400a (e.g., a microprocessor), at least one permanent memory module 400b (e.g., ROM), at least one transient memory module (e.g., RAM) 400c, and a bus 400d, among other elements generally known in the art.

The bus 400d allows communication between the processor and various other components of the controller. In this example, the controller 400 can further include a communications module 400e that is configured to allow sending and receiving signals.

Instructions for use by the controller 400, e.g., for operating the pump 1018, can be stored in the permanent memory module 400b and can be transferred into the transient memory module 400c during runtime for execution. The controller 400 can also be configured to control the operation of other components of the mass spectrometer, such as the ion guide, and mass analyzer, among others.

The methods and systems according to the present teachings can be employed for analysis of a variety of different target analytes, including those in biological matrices. For example, the methods and systems can be employed for analysis of a plurality of target analytes in a complex biological matrix. Some examples of such biological samples include, without limitation, saliva, urine, whole blood, plasma, serum, cell matrix, among others. It should, however, be understood that the application of methods and systems according to the present teachings are not limited to biological samples, but could be utilized for mass analysis of a variety of analytes contained in a variety of different matrices.

The following examples are provided for further elucidation of various aspects of the present teachings, and are not provided to indicate necessarily optimal ways of practicing the present teachings and/or optimal results that may be obtained.

### Examples

In one series of experiments, mass analysis of dextromethorphan in urine was conducted with and without extraction of the dextromethorphan into an organic phase, as discussed below. In all experiments, a SCIEX Triple QUAD 6500+mass spectrometer was employed to measure the 272.2→215.1 MRM transition of dextromethorphan in urine. While in this example, a single MRM transition is monitored, in other cases, a plurality of MRM transitions may be monitored.

The mass spectrometer included an open port interface (OPI) and an acoustic actuator for ejecting one or more droplets from a sample into the OPI of the mass spectrometer.

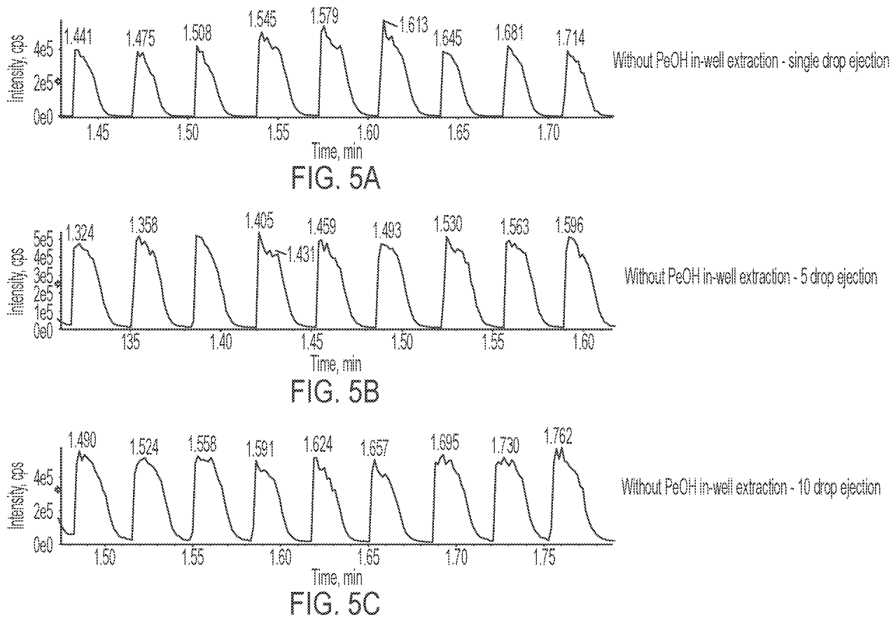

FIGS. 5A, 5B, and 5C show a series of measurements of the above MRM transition of dextromethorphan that were acquired, respectively, via the ejection of a single droplet, 5 droplets and 10 droplets of the urine sample into the mass spectrometer without using organic phase extraction in accordance with the present teachings.

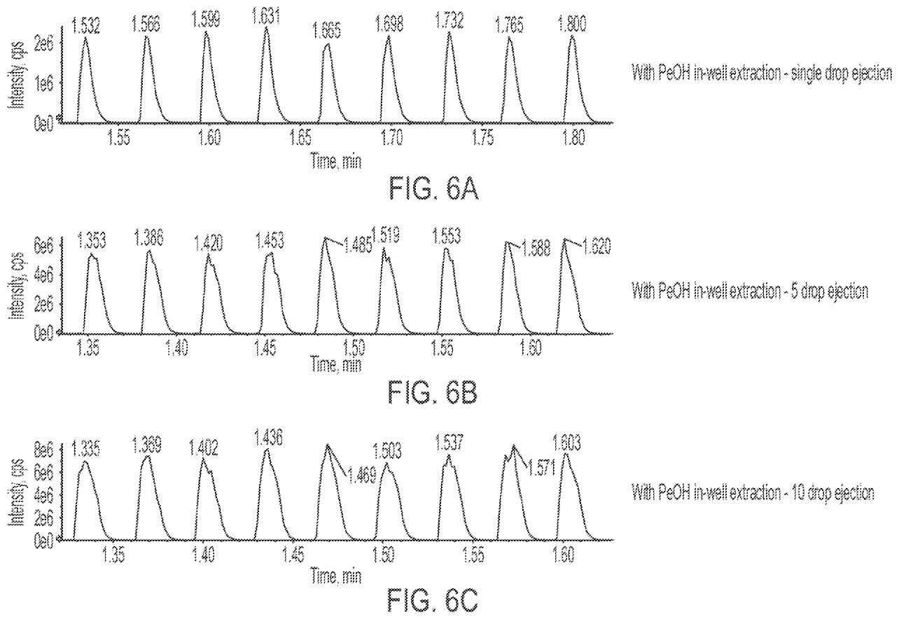

In another series of experiments, each of several samples of a two-phase system was prepared by adding 20 μL of pentanol (PeOH) to 25 μL of a urine sample. FIGS. 6A, 6B, and 6C show a series of measurements of the above MRM transition of dextromethorphan that were acquired, respectively, via ejection of a single droplet, 5 droplets and 10 droplets from the organic phase of the two-phase system into the mass spectrometer.

A comparison of the mass data presented in FIGS. 5A, 5B, and 5C with the mass data presented, respectively, in FIGS. 6A, 6B, and 6C shows that a significant enhancement of the mass signals can be obtained when using the two-phase systems. Without being limited to any particular theory, the extraction of dextromethorphan from the urine matrix into the pentanol layer can enhance the mass signal, e.g., via significant reduction of ionization suppression.

Although some aspects have been described in the context of a system and/or an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware and/or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

It should be understood that the methods and systems according to the present teachings can be employed for predicting the cleavage products of a variety of macromolecules.

What is claimed is:

1. A method of introducing a sample into a mass spectrometer, comprising:
mixing the sample with at least one solvent in which an aqueous matrix of an aqueous phase of said sample is immiscible and in which at least a target analyte, when present in said sample, is miscible so as to extract at least a portion of said at least one target analyte into said at least one solvent, thereby generating a multi-phase liquid having said aqueous phase and one or more organic phases, wherein at least one of said organic phases contains said at least one target analyte, and
introducing one or more samples from at least one of the phases of the multi-phase liquid into the mass spectrometer.

2. The method of claim 1, wherein said step of introducing one or more samples comprises introducing one or more samples of said organic phase containing said at least one target analyte into the mass spectrometer.

3. The method of claim 1, wherein said step of introducing said at least one of the phases comprises ejecting a plurality of droplets from said at least one phase containing said target analyte into the mass spectrometer.

4. The method of claim 3, further comprising using an acoustic actuator for ejecting said plurality of droplets.

5. The method of claim 1, wherein said at least one solvent comprises a first and a second solvent and said at least one target analyte comprises a first and a second analyte, wherein said first analyte is miscible in said first solvent and said second analyte is miscible in said second solvent.

6. The method of claim 1, wherein said one or more organic phases comprise two organic phases.

7. The method of claim 6, wherein said two organic phases comprise hexane and methyl acetate and said aqueous phase comprises acetonitrile and water.

8. The method of claim 7, wherein a relative concentration ratios of said hexane, said methyl acetate, said acetonitrile and said water is 4:4:3:4.

9. The method of claim 1, wherein at least one of said organic phases is more dense than said aqueous phase.

10. The method of claim 1, wherein said aqueous phase is more dense than at least one of said organic phases.

11. The method of claim 10, wherein said at least one of said organic phases comprises any of an alcohol, chloroform, dichloromethane, and ethyl ether.

12. The method of claim 11, wherein said alcohol comprises any of pentanol and octanol.

13. A system for introducing a sample into a mass spectrometer, comprising:
a first reservoir for storing a sample containing or suspected of containing one or more analytes,
a second reservoir for storing a solvent in which an aqueous matrix of said sample is immiscible and said one or more analytes are miscible,
a mechanism for generating a flow of said solvent from said second reservoir into said first reservoir so as to mix said solvent with said sample so as to extract at least a portion of said one or more analytes into said solvent, thereby generating a two-phase solution having a first phase comprising said aqueous matrix and a second phase comprising a mixture of said solvent and at least a portion of said one or more analytes, and
a mechanism operably coupled to said first reservoir for ejecting a plurality of droplets from any of said first phase and second phase for introduction into said mass spectrometer.

14. The system of claim 13, wherein said mechanism is configured to apply one or more acoustic pulses to any of said first and said second phase for generating said droplets.

15. The system of claim 13, wherein said solvent comprises any of an alcohol, chloroform, dichloromethane, and ethyl ether.

16. The system of claim 13, wherein said mechanism for generating the flow of the solvent comprises a pump fluidly coupled to said first and second reservoir.

17. The system of claim 16, further comprising a controller operably coupled to said pump for controlling thereof.

18. The system of claim 17, wherein said controller controls the pump so as to cause transfer of a predefined volume of said solvent from said second reservoir to said first reservoir.

19. The system of claim 13, wherein said mechanism for ejecting the plurality of droplets comprises an acoustic actuator.

20. The system of claim 13, wherein said mass spectrometer comprises an open port interface (OPI) for receiving said plurality of droplets.
